(19) United States
(12) Patent Application Publication
(10) Pub. No.: US 2022/0413140 A1
(43) Pub. Date: Dec. 29, 2022

(54) RANGE INFORMATION DETECTION USING COHERENT PULSE SETS WITH SELECTED WAVEFORM CHARACTERISTICS

(71) Applicant: Seagate Technology LLC, Fremont, CA (US)

(72) Inventor: Daniel Joseph Klemme, Robbinsdale, MN (US)

(21) Appl. No.: 17/833,066

(22) Filed: Jun. 6, 2022

Related U.S. Application Data
(60) Provisional application No. 63/216,206, filed on Jun. 29, 2021.

(51) Int. Cl. G01S 17/10; G01S 7/484

(52) U.S. Cl. CPC G01S 17/10; G01S 7/484

(57) ABSTRACT

Method and apparatus for obtaining range information associated with a target using light detection and ranging (LiDAR). An emitter transmits a set of pulses of electromagnetic radiation to illuminate a target. The set of pulses includes a pair of emitted pulses with different waveform characteristics, such as slightly different phases. A detector receives a reflected set of pulses from the target. The received set of pulses includes a pair of received pulses with corresponding different waveform characteristics. The detector determines the range information by decoding the received pulses, such as by calculating an average of the phase differential in the received pulses. In this way, a single stage detector can be used without the need for separate I/Q (in-phase and quadrature) channels. Phase chirping can be used so that each successive pair of pulses has a different phase difference. Other waveform characteristics can be used including frequency, amplitude, shape, etc.

### RELATED APPLICATIONS

The present application makes a claim of domestic priority under 35 U.S.C. 119(e) to U.S. Provisional Patent Application No. 63/216,206 filed Jun. 29, 2021, the contents of which being hereby incorporated by reference.

### SUMMARY

Various embodiments of the present disclosure are generally directed to a method and apparatus for obtaining range information associated with a target using light detection and ranging (LiDAR) techniques.

Without limitation, in some embodiments an emitter is used to emit a set of pulses of electromagnetic radiation to illuminate a target. The set of pulses includes a pair of emitted pulses with different waveform characteristics, such as slightly different phases. A detector receives a reflected set of pulses from the target. The received set of pulses includes a pair of received pulses with corresponding different waveform characteristics. The detector determines the range information by decoding the received pulses, such as but not limited to determining an average of the phase differential in the received pulses.

In this way, a single stage detector can be used without the need for separate I/Q (in-phase and quadrature) channels. Phase chirping can be used so that each successive pair of pulses in a cycle has a different phase difference. Other waveform characteristics can be used including frequency, amplitude, shape, etc.

These and other features and advantages of various embodiments can be understood from a review of the following detailed description in conjunction with the accompanying drawings.

### BRIEF DESCRIPTION OF THE DRAWINGS

FIG. 1 is a block representation of a Light Detection and Ranging (LiDAR) system constructed and operated in accordance with various embodiments of the present disclosure.

FIG. 2 shows an emitter of the related art.

FIG. 3 shows a detector of the related art.

FIG. 4 shows an emitter in accordance with some embodiments.

FIG. 5 graphically illustrates pulses that can be generated by the emitter of FIG. 4.

FIG. 6 shows a detector in accordance with some embodiments.

FIG. 7 is a functional block representation of a pulse detection and analysis circuit constructed and operated in accordance with some embodiments.

FIG. 8 is a sequence timing diagram to illustrate operation of the circuit of FIG. 7 in some embodiments.

FIG. 9 graphically depicts a number of pulse waveforms that can be generated and used in accordance with various embodiments.

FIG. 10 is a sequence diagram to set forth a range information detection operation carried out in accordance with various embodiments.

FIG. 11 shows another detector in accordance with further embodiments.

FIG. 12 shows another arrangement of a LiDAR system in accordance with further embodiments.

### DETAILED DESCRIPTION

Various embodiments of the present disclosure are generally directed to systems and methods for detecting a target using specially configured coherent light pulses.

Light Detection and Ranging (LiDAR) systems are useful in a number of applications in which range information (e.g., distance) associated with a target is detected by irradiating the target with electromagnetic radiation in the form of light. The range information is detected in relation to timing characteristics of reflected light received back by the system. LiDAR applications include topographical mapping, guidance, surveying, and so on. One increasingly popular application for LiDAR is in the area of autonomously piloted or driver assisted vehicle guidance systems (e.g., self driving cars, autonomous drones, etc.). While not limiting, the light wavelengths used in a typical LiDAR system may extend from ultraviolet to near infrared (e.g., 250 nanometers, nm to 1000 nm or more).

One commonly employed form of LiDAR is sometimes referred to as coherent pulsed LiDAR, which generally uses coherent light and detects the range based on detecting phase differences in the reflected light. Such systems often use a dual (I/Q) channel detector with an I (in-phase) channel and a Q (quadrature) channel. While operable in providing precise measurements, such systems can be relatively complex, requiring one or more mixers, filters and other components in each detection channel.

Accordingly, various embodiments of the present disclosure are generally directed to a method and apparatus for providing a coherent LiDAR detection system with simplified detection arrangements.

As explained below, at least some embodiments involve the application of phase chirping to each of at least two pulses that are issued in close succession toward the target, each having slightly different phases. For example, a first pulse may be supplied with a slightly increased (chirped up) phase and a second pulse may be supplied with a slightly decreased (chirped down) phase. The succession of pulses are directed to the target and the system monitors for detected, reflected pulses. The phase differentials may be slowly adjusted during the transmission and detection operation. Other differences in waveform characteristics can be applied to the successive pulses.

In this way, both received pulses will have at least some time on the detector, and the average (or some other calculated combination) of those two detected pulses can be used to generate the true range to the target using a single detection channel, thereby eliminating the need for separate I/Q channels.

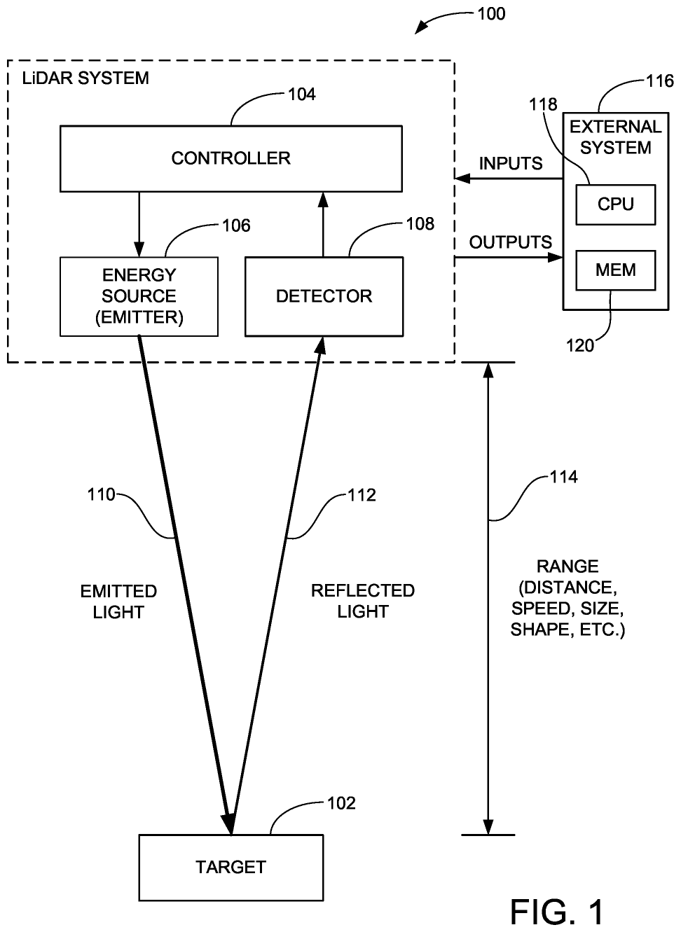

These and other features and advantages of various embodiments can be understood beginning with a review of FIG. 1, which provides a simplified functional representation of a LiDAR system 100 constructed and operated in accordance with various embodiments of the present disclosure. The LiDAR system 100 is configured to obtain range information regarding a target 102 that is located distal from the system 100. The information can be beneficial for a number of areas and applications including, but not limited to, topography, archeology, geology, surveying, geography, forestry, seismology, atmospheric physics, laser guidance, automated driving and guidance systems, closed-loop control systems, etc.

The LiDAR system 100 is shown to include a controller 104 which provides top level control of the system. The controller 104 can take any number of desired configurations, including hardware and/or software. In some cases, the controller can include the use of one or more programmable processors with associated programming (e.g., software, firmware) stored in a local memory which provides instructions that are executed by the programmable processor(s) during operation. Other forms of controllers can be used, including hardware based controllers, digital signal processors (DSPs), field programmable gate arrays (FPGAs), system on chip (SOC) integrated circuits, application specific integrated circuits (ASICs), gate logic, reduced instruction set computers (RISCs), etc.

An energy source circuit 106, also sometimes referred to as an emitter, operates to direct electromagnetic radiation in the form of light towards the target 102. A detector circuit 108 senses reflected light that is received back from the target 102. The controller 104 directs operation of the emitted light from the emitter 106, denoted by arrow 110, and decodes information from the reflected light obtained back from the target, as denoted by arrow 112.

Arrow 114 depicts the actual, true range information associated with the intervening distance (or other range parameter) between the LiDAR system 100 and the target 102. Depending on the configuration of the system, the range information can include the relative or absolute speed, velocity, acceleration, distance, size, location, reflectivity, surface features and/or other characteristics of the target 102 with respect to the system 100. Optimally, the system 100 is configured to be able to determine, with high levels of accuracy, the true range information (also referred to as range data).

The decoded range information can be used to carry out any number of useful operations, such as controlling a motion, input or response of an autonomous vehicle, generating a topographical map, recording data into a data structure for further analysis and/or operations, etc. The controller 104 perform these operations directly, or can communicate the range information to an external system 116 for further processing and/or use.

In some cases, inputs supplied by the external system 116 can activate and configure the system to capture particular range information, which is then returned to the external system 116 by the controller 104. The external system can take any number of suitable forms, and may include a system controller (such as CPU 118), local memory 120, etc. The external system may form a portion of a closed-loop control system and the range information output by the LiDAR system 100 can be used by the external system 116 to adjust the position of a moveable element.

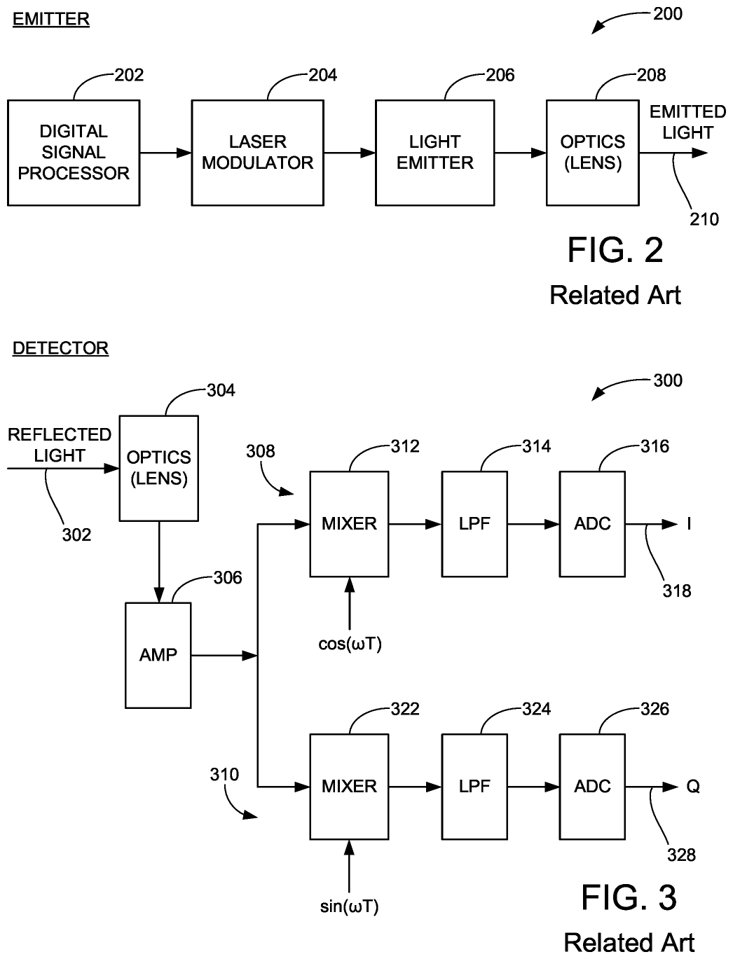

To better set forth aspects of the present disclosure, FIGS. 2-3 have been provided to illustrate some types of emitter and detector circuitry that have been used in accordance with the existing art. FIG. 2 shows an emitter 200 of the related art, and FIG. 3 shows a corresponding detector 300 of the related art.

In FIG. 2, The emitter 200 is generally characterized as a coherent light emitter, and operates to detect range information such as depicted in FIG. 1 using collated light at a selected wavelength (e.g., infrared, visible, ultraviolet, etc.).

The emitter 200 includes a digital signal processor (DSP) 202 which provides inputs to a laser modulator circuit 204. The laser modulator circuit 204, in turn, drives a light emitter 206 which may be an electromagnetic radiation light source such as a light emitting diode (LED) or laser. The light emitter 206 outputs light at a selected frequency and wavelength.

The output of the light emitter 206 is directed through a set of optics (e.g., an optical lens, etc.) to generate emitted light 208 that is transmitted downrange toward a target in a manner similar to that described in FIG. 1. The light may be in the form of continuous light, discrete pulses, etc. Mechanical or solid-state mechanisms (e.g., a phased array, a rotatable mirrored polygon, etc.) can be used to direct the light in a selected direction in order to detect the downrange target.

The detector 300 in FIG. 3 is used to subsequently detect the light emitted by the emitter 200 in FIG. 2 that is reflected from the target. The detector 300 is characterized as an I/Q channel detector. I/Q is an abbreviation for “in-phase” and “quadrature,” respectively, and this generally describes two sinusoids having the same frequency and which are 90 degrees out of phase. This 90 degree phase differential is referred to as a quadrature relation. By convention, the I signal is sometimes referred to as a cosine waveform, and the Q signal as a sine waveform.

The detector 300 is arranged to receive reflected light, denoted at 302, from the target. The reflected light 302 is processed by receiving optics 304, such as one or more lenses, and forwarded to an amplifier (amp) 306. At this point the received input is directed to two separate and parallel processing channels, referred to respectively as an I channel 308 and a Q channel 310.

The I channel 308 includes a mixer circuit 312 which combines the received signal with an input based on a selected function, such as a cosine function, which in turn is based on a base reference signal (wT). This is merely for purposes of illustration and is not limiting, as other coherent detection systems are known in the art.

The output of the mixer 312 is supplied to a low pass filter (LPF) 314 and an analog to digital converter (ADC) 316 to provide an output I, denoted at 318.

The Q channel 310 generally operates in a similar fashion to provide a corresponding output Q that is nominally 90 degrees out of phase with respect to the data supplied along the I channel. As before, the Q channel 310 includes a mixer 322 which combines the input signal with a different reference based on a selected function, such as in this case a sine function again in relation to the base reference signal (ωT). An LPF 324 and ADC 326 produce the corresponding output signal Q, denoted at 328. By combining the respective quadrature (orthogonal) I and Q channel outputs, full spectra information can be gained from the system and a nominally accurate determination of the range information can be determined using a processing circuit, such as the DSP 202 in FIG. 2.

While operable, these and other forms of LiDAR systems require significant investments and resources due to the circuit complexity required to support, and thereafter combine, the separate I and Q channels.

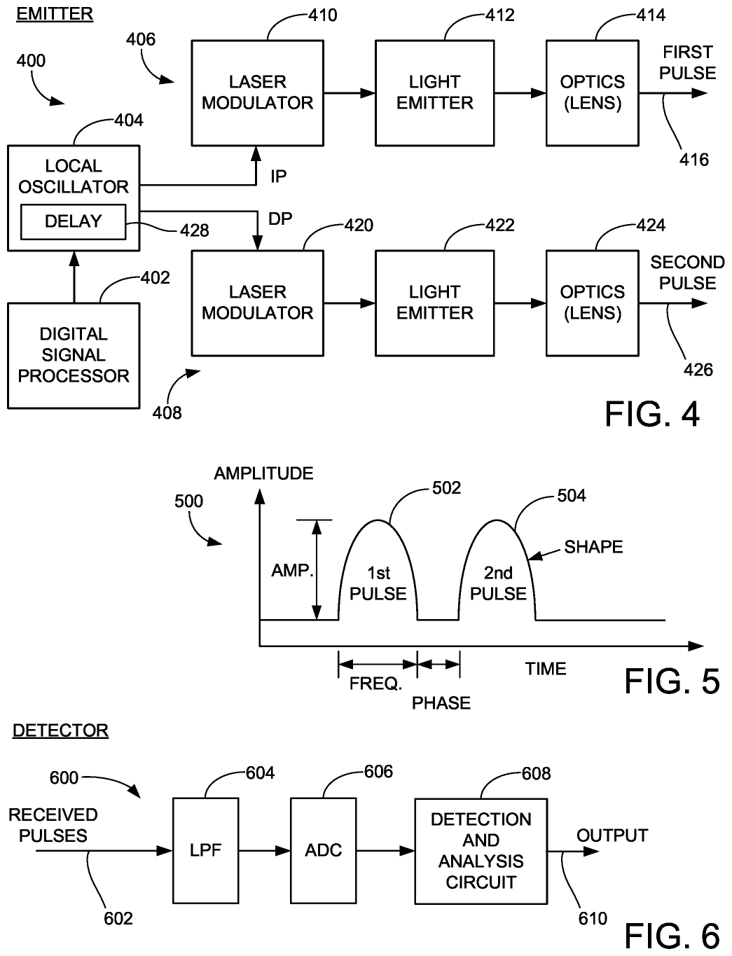

Various embodiments of the present disclosure are directed to improvements in the art such that the same or improved levels of performance can be obtained without the necessity of providing separate I and Q channels as in FIG. 3. To this end, FIG. 4 depicts an emitter 400 constructed and operated in accordance with various embodiments of the present disclosure. It will be understood that the functional representation in FIG. 4 is merely illustrative, as other configurations can be used. The circuitry 400 in FIG. 4 forms at least a portion of the emitter 106 in FIG. 1 in some embodiments.

The emitter 400 includes a digital signal processor (DSP) 402 which provides selected inputs to a local oscillator 404, which is configured to output different outputs and different frequencies and phases. The local oscillator 404 drives two separate and parallel emission channels 406, 408. These two channels 406, 408 are sometimes referred to as a first channel and a second channel, respectively. While two separate channels 406, 408 are shown in the embodiment of FIG. 4, in other embodiments the operations described below can be accomplished using a single emission channel that operates successively in different time frames. Conversely, other embodiments can be configured to use three or more separate emission channels.

The first channel 406 includes a laser modulator circuit 410 which directs a light emitter 412 to direct, via an optics (e.g. lens) arrangement 414, at least a first pulse 416 having a first set of waveform characteristics, such as a selected wavelength, a selected phase, a selected frequency, a selected amplitude, a selected shape, etc.

The second channel 408 similarly includes a laser modulator 420 which directs a light emitter 422 to direct, via a corresponding optics arrangement 424, a second pulse having a different, second set of waveform characteristics. At least one waveform characteristic will be different between the two respective sets.

The two channels 406, 408 are driven in parallel from the local oscillator 404 via slightly different input signals. In one embodiment, an in-phase (IP) modulation pulse is forwarded to the laser modulator 410 and a delayed phase (DP) modulation pulse is forwarded to the laser modulator 412 a short time after the IP modulation pulse. This can be achieved in a number of ways, including through the use of a fixed or tunable delay circuit 428 that delays the IP pulse to provide the DP pulse.

It is contemplated albeit not necessarily required that the emitter 400 will emit successive sets of pulses, such as the pair of pulses 416/426, each having slightly different waveform characteristics. In one embodiment, the two pulses each are at the nominally same frequency (or wavelength) but are at slightly different phases. Other options can be provided, including providing the successive pulses with slightly different frequencies, wavelengths, amplitudes, etc.

FIG. 5 is a waveform 500 plotted against an elapsed time x-axis and an amplitude y-axis. The waveform 500 includes a first pulse 502 and a second pulse 504. The first pulse 502 is generated by the channel 406 in FIG. 4 and has a first (e.g., baseline) phase. The second pulse 504 is generated by the channel 408 in FIG. 4 and has a second (e.g., delayed) phase. While two successive pulses are depicted, it will be appreciated that any plural number of pulses can be emitted as a set of pulses in accordance with various embodiments.

As represented in FIG. 5, the pulses will have various tuned waveform characteristics including amplitude (e.g., pulse height), frequency (pulse width), phase (in terms of separation distance/time between pulses), shape (sinusoid, square, trapezoidal, triangular, irregular, etc.), and substantially any other characteristic as desired. It will be noted that sinusoids are depicted for clarity of illustration, but substantially any style pulses can be used.

The DSP 402 in FIG. 4 can be configured to cause the local oscillator 404 to vary, over time, the differences in phase changes (or other distinctive waveform characteristics) of the sets of pulses sent out by the emitter 400. In some embodiments, a chirping operation is used so that some pulses are provided with slightly higher phases and other pulses are provided with slightly lower phases. The relative differences are varied to provide a range over which the pulses are swept. This can be provided repetitively on a cyclical basis.

FIG. 6 shows a detector circuit 600 constructed and operated in accordance with various embodiments. The detector circuit 600 forms at least a portion of the detector 106 of FIG. 1 in some embodiments. The detector 600 operates to detect and decode the pulses generated and emitted by the emitter 400 of FIG. 4 to derive range information regarding the downrange target. The arrangement in FIG. 6 is merely illustrative and is not limiting, as other arrangements can be used as desired.

The detector circuit 600 receives as an input a sequence of received pulses 602 that are reflected from the associated target. Initial processing can be supplied to these received pulses as described above in FIG. 3, such as channeling of the reflected light using a suitable optics assembly, conditioning of the detected signals using an amplifier, etc.

An LPF 604 applies low pass filtering over a frequency range of interest to reduce noise and other undesired components. An ADC 606 provides analog to digital conversion as required. It is contemplated that, when used, the ADC 606 provides sufficient granularity to precisely and accurately capture the characteristics of interest in the received pulses.

A detection and analysis circuit 608 takes the output pulses from the upstream components 604, 606 and applies a suitable analysis function to obtain the desired range information without the need for separate I and Q channels as in FIG. 3. In some embodiments, the analysis function is an averaging function so that the average power, phase, or other characteristic is monitored and used. The phase differentials can be slowly adjusted over successive sets of pulses to enable the detector to determine the true range information associated with the target. The differentials in the sets of pulses can be used to match particular pulse sets. Changes in the received pulse sets can also be used to determine the range information.

The circuit 608 can be a separate dedicated circuit or can form a portion of a DSP (such as 402 in FIG. 4) or other controller circuitry. The processing carried out by the circuit 608 can include a variety of functions including averaging, weighting, subtraction, comparison, and/or other combinatorial operations.

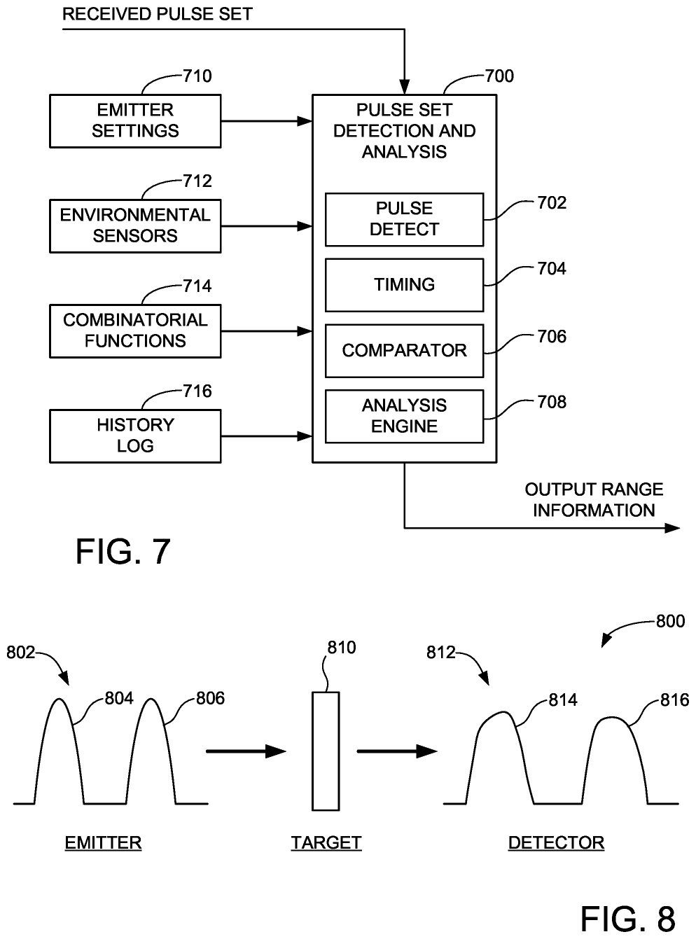

FIG. 7 provides a functional block representation of another detector circuit 700 constructed and operated in accordance with various embodiments. Other arrangements can be used. Aspects of the circuit 700 can be incorporated into the detection circuits 108, 600 discussed above. While not limiting, the circuit 700 is contemplated as incorporating one or more programmable processors that have associated programming to enact the various functions that will now be described.

The circuit 700 includes a pulse detector circuit 702, a timer circuit 704, a comparator circuit 706, and an analysis engine 708. Upon receipt of each set of pulses from the target, the detector circuit 700 characterizes, such as via measurements, calculations, etc., various characteristics of each pulse, such as those depicted in FIG. 5. The timer circuit 704 provides baseline timing and counting functions and may utilize a high speed clock circuit (not separately shown). Synchronization may be maintained with the emitter 400 (FIG. 4) for timing accuracy purposes.

The comparator circuit 706 analyzes the extracted information regarding the respective pulses in each set, and the analysis engine 708 uses this information to arrive at an accurate indication of the range information regarding the target.

In some embodiments, the circuit 700 can utilize external inputs as part of the detection and analysis operation. Such inputs can include but are not limited to emitter setting information from an emitter setting circuit 710, environmental sensors 712, various available combinatorial functions from circuit 714, and history data regarding previous detections from a history log 716. Other suitable inputs can be supplied and used as well. The emitter settings 710 may include timing, frequency, phase, pulse count, and other information regarding the transmitted pulse sets from the emitter.

The manner in which the circuit 700 decodes range information from sets of received pulses can be understood with a review of FIG. 8, which is a simplified graphical flow diagram of a pulse transmission and reflection sequence 800.

An initial set of pulses is depicted at 802. This initial set 802 has two pulses 804, 806. Both pulses are at the same nominal frequency and amplitude, and at a selected phase difference as established by the emitter 400 in FIG. 4.

The emitted pulses are quanta of electromagnetic energy that are transmitted downrange toward a target 810. Reflected off of the target is a received set of pulses 812, which has two corresponding pulses 814, 816.

A comparison of the respective pulses 802/804 and 814/816 shows that changes have been induced as a result of interaction with the target as well as the intervening distances (both transmitting and reflecting) between the emitter/detector and the target. It will be noted that the changes shown in FIG. 8 have been exaggerated for clarity. Changes can include differences in amplitude, phase, frequency, shape, etc. These changes can be correlated to the various types of range information discussed above. Other changes between the respective pulse sets can be induced as a result of system noise and effects, but such can be systemic and can be accounted and compensated for including through calibrations, adjustments to account for sensed environmental conditions, etc. It will be appreciated that the particular characteristics of both emitted and received pulses may tend to vary depending on operational settings and environmental factors.

For example and not by way of limitation, the elapsed time interval from emission of the first pulse set 802 to detection of the second pulse set 812, based on the speed of light (as compensated for by medium effects as required) can provide an accurate indication of distance to the target. Averaging of the pulses 814 and 816 can be used as part of this analysis. The use of multiple pulses, chirped pulses at different frequencies and/or phases, etc. can facilitate matching of emitted and received sets and detect changes over time to derive velocity, direction and other vector information associated with the target relative to the detector.

Frequency and phase changes in the received pulses (e.g., both between the individual pulses 814, 816 as well as differences between the received pulses 814/816 and the emitted pulses 804/806) can be used part of the decoding operation. In some embodiments, changes in shape and spectral components in the received pulses can be used to provide further information regarding the target (e.g., color, reflectivity, texture, etc.). The extraction of the range information from the reflected pulses can be based on analytical and/or empirical operations that provide reference and/or calibration table data sets used in the decoding process.

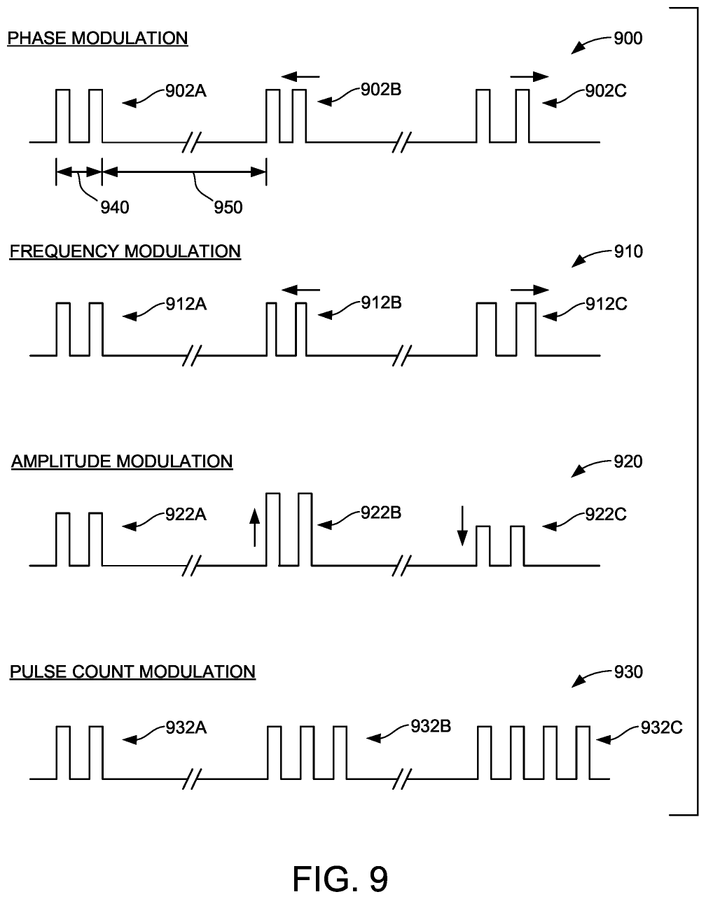

FIG. 9 shows different types of modulation can be applied to various pulse sets depending on the requirements of a given application. Square-wave (PWM) pulses are depicted in these examples, although such is not limiting.

A first waveform 900 depicts phase modulation, or chirping, in which successive pulse sets have different, controlled amounts of phase differentials. The first waveform 900 provides a first pulse set 902A with a baseline phase difference, a second pulse set 902B with a smaller phase difference and a third pulse set with a larger phase difference.

As noted previously, a pulse set cycle can be generated as a succession of pulse sets that are sent as a unit, after which the cycle is repeated continuously. Some embodiments begin with a first phase differential for the first pulse set and then slowly increase or decrease the phase in each subsequent pulse set in the pulse set cycle. The elapsed time between pulse sets can be set to an appropriate interval. This intervening interval between successive pulse set pairs should be of sufficient length to enable the system to distinguish among the respective pulse sets. The interval between the pulse sets can remain nominally constant at a fixed value or can be varied by a selected amount for each successive pairs of pulse sets in the cycle.

Control inputs supplied to the local oscillator (404 in FIG. 4) can be used to generate the requisite modulation signals necessary to output the pulses with the desired characteristics. It will be noted that in at least some embodiments the phase differentials in each pulse set are specifically limited so as to not be zero and to not be some multiple of 90 degrees (e.g., something other than 0, 90, 180, 270, etc.). Stated another way, the pulses in each pulse set are non-quadrature pulses. The actual phase differentials in the waveform 900 can vary; examples include +/−5 degrees, +/−10 degrees, +/−20 degrees, +/−30 degrees, etc. In one non-limiting embodiment, the phase differential magnitude is nominally between 5 degrees and 30 degrees. Other ranges can be used.

While phase modulation is contemplated as a particularly suitable embodiment, other forms of modulation can additionally or alternatively be used to obtain effective results. Waveform 910 in FIG. 9 shows frequency modulation in which different frequencies are applied to the pulses, as denoted by respective pulse sets 912A, 912B and 912C. In this case, the pulse sets nominally maintain the same phase differential but are provided with different widths.

Waveform 920 depicts amplitude modulation so that different pulse sets are provided with different amplitudes, such as via pulse sets 922A, 922B and 922C. Waveform 930 shows pulse count modulation so that different pulse sets have different numbers of pulses, such as shown by pulse sets 932A, 932B and 932C. These and other types of modulation can be combined as required; for example, phase modulation can be combined with pulse count modulation, etc.

From FIG. 9 it can be seen that each of the exemplary waveforms 900, 910, 920, 930 has a first pulse set (e.g., 902A) and a number of additional pulse sets (e.g., 902B, 902C). Each pulse set occurs over an associated pulse set time interval which can be viewed as the elapsed time from the leading edge of the first pulse to the trailing edge of the second pulse in a given pulse set. One such pulse set time interval is denoted at 940 for pulse set 902A.

As noted above, an intervening elapsed time interval is provided from each successive pair of pulse sets in each waveform. One such intervening elapsed time interval is denoted at 950, which is the elapsed time between successive pulse sets 902A and 902B. In many cases, the intervening time interval 950 will be multiple times in duration as compared to the pulse set interval 940 (e.g., interval 950 will be 3X, 5X, 10X, etc. longer than interval 940). Other ranges can be used. Generally, the intervening duration between successive pulse sets will be sufficiently long to enable detection and decoding of the individual pulse sets.

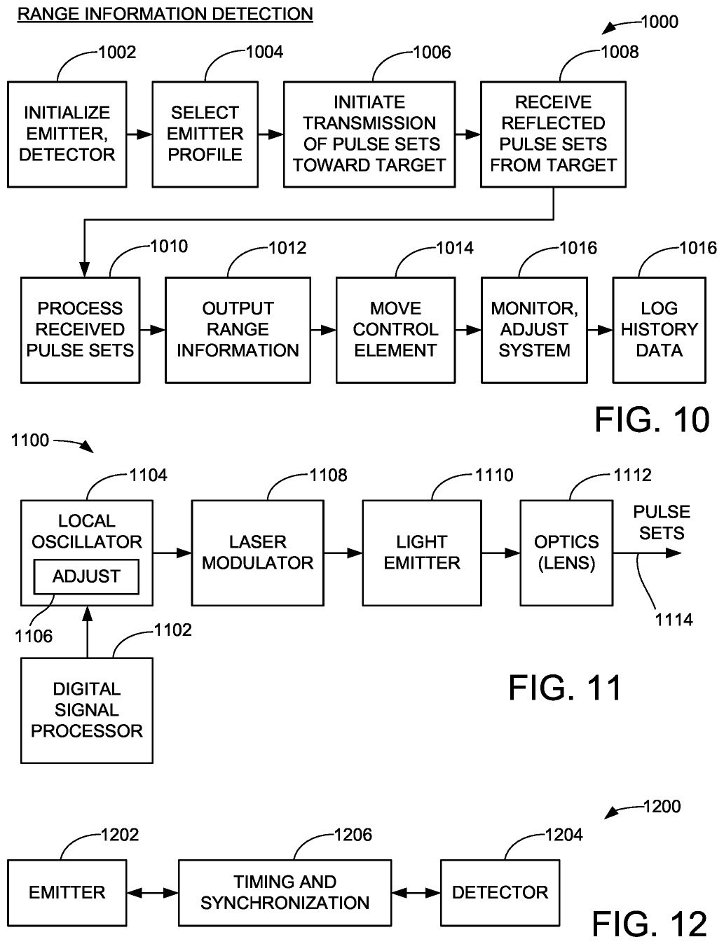

FIG. 10 shows a sequence diagram 1000 to illustrate range information detection that can be carried out in accordance with the various embodiments presented herein. The sequence can be modified, appended, carried out in a different order, etc., depending on the requirements of a given application.

A light detection and ranging (LiDAR) system is configured with an emitter and a detector as described above, and the system is initialized at block 1002. The initialization can be carried out internally by the controller (104, FIG. 1) or responsive to an input configuration signal from an external source (116, FIG. 1).

A suitable emitter profile is selected at block 1004. The profile will include, inter alia, a particular arrangement of coherent pulses to be emitted by the emitter (e.g., 104, 400). This can include selection of an appropriate pulse set cycle configuration to be repetitively output by the emitter. The system can be configured to operate continuously once activated, or to operate for a predetermined period of time.

The system proceeds at block 1006 to initiate the transmission downrange of the pulse sets from the emitter in accordance with the selected profile. As noted previously, phase array or mechanical directional systems can be utilized to direct the pulses in a desired direction or over a selected angular window.

At least some of the emitted pulse sets will be reflected from the target and received by the detector at block 1008. These pulse sets are processed at block 1012 as described above by the detector (e.g., 106, 600, 700) to generate and output range information associated with the target. Further operations can be carried out such as making an adjustment to the position of a control element (block 1014), monitoring and adjusting the system (block 1016), and accumulating log history data (block 1018).

A dual channel emitter 400 was described above in FIG. 4 to facilitate the generation and outputting of the respective pulses in each pulse set. In this prior example, each pulse set had two pulses, and each pulse was generated by a different channel. FIG. 11 shows an alternative embodiment for a single channel emitter 1100 in accordance with further embodiments. The emitter 1100 has several components similar to the emitter 400, but uses a single channel to generate all of the pulses in each pulse set.

The emitter includes a DSP 1102, local oscillator 1104 with high speed adjustment circuit 1106, laser modulator 1108, light emitter 1110 and optical system 1112. Provided the oscillator, modulator and light emitter can respond at a sufficient rate to change the output characteristics of the emitted pulses, any number of different pulse set profiles can be generated and outputted using the same channel.

FIG. 12 provides another LiDAR system 1200 in accordance with further embodiments. The system 1200 is similar to the systems described above in FIGS. 1 and 4-11. The system 1200 includes an emitter 1202 and a detector 1204 which operate in accordance with the sequence diagram of FIG. 10.

A timing and synchronization circuit 1206 is provisioned between the respective emitter 1202 and detector 1204. The circuit 1206 controls the timing and synchronization of pulses so that the detector, upon detecting a particular pulse set, can correlate the received pulse set to the corresponding pulse set that was transmitted by the emitter. In this way, the circuit 1206 tracks which pulse sets are being emitted, and the detector can generate the range information based on either or both the timing differences between the emitted/received pulse sets as well as based on characteristics of the received pulses themselves. The circuit 1206 can be a dedicated circuit or can be realized via programming, such as a routine executed by the DSP or other programmable processor.

It will now be understood that the various embodiments presented herein provide a number of benefits over the existing art. A coherent LiDAR emitter can be used to supply pulses in sets with different phases (or other waveform characteristics) in a selected relation to one another. The differences among the pulses can be decoded to supply true range information without the need to establish in phase and quadrature I/Q detector channels, as in the existing art.

It is to be understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. A method, comprising:
emitting, from an emitter, a set of pulses of electromagnetic radiation to illuminate a target, the set of pulses comprising a first emitted pulse with a first waveform characteristic and a second emitted pulse with a second waveform characteristic different from and in non-quadrature relation with the first waveform characteristic;
receiving, by a detector, a reflected set of pulses from the target, the reflected set of pulses comprising a first received pulse corresponding to the first emitted pulse and a second received pulse corresponding to the second emitted pulse; and
combining the first received pulse with the second received pulse to determine range information associated with the target.

2. The method of claim 1, wherein the first and second waveform characteristics are each a phase of the respective first and second emitted pulses, wherein the second emitted pulse is out of phase with the first emitted pulse by a non-zero phase differential that is not a multiple of 90 degrees, and the range information is determined responsive to a detected phase differential between the first and second received pulses.

3. The method of claim 2, wherein the non-zero phase differential between the first and second emitted pulses has a magnitude of nominally between 5 degrees and 30 degrees.

4. The method of claim 1, wherein the range information is determined responsive to an average of respective phases of the first and second received pulses.

5. The method of claim 1, wherein the first and second waveform characteristics are each a frequency of the respective first and second emitted pulses so that the first emitted pulse is at a first frequency and the second emitted pulse is at a different, second frequency.

6. The method of claim 1, wherein the first and second waveform characteristics are each an amplitude of the respective first and second emitted pulses, and wherein the first emitted pulse has a first amplitude and the second emitted pulse has a different, second amplitude.

7. The method of claim 1, wherein a first modulation signal is applied to a first light source to generate the first emitted pulse, and wherein the first modulation signal is further applied to a delay circuit to generate a delayed modulation signal which is applied to a second light source to generate the second emitted pulse, the first and second light sources arranged in separate, parallel channels of an emitter.

8. The method of claim 1, wherein the combining step comprises evaluating a difference in at least a selected one of frequency, waveform shape, phase differential or amplitude between the first and second received pulses to determine the range information.

9. The method of claim 1, wherein the set of pulses is a first pulse set having the first and second emitted pulses, and wherein the method further comprises successively emitting each of a plurality of additional pulse sets each comprising at least corresponding first and second emitted pulses, each of the additional pulse sets having at least a selected one of a different average phase differential, a different average wavelength, a different average amplitude or a different overall total number of pulses therein.

10. The method of claim 9, wherein each of the first pulse set and the additional pulse sets occur over an associated pulse set time interval, wherein an intervening elapsed time interval is provided between each successive pair of the first pulse set and the additional pulse sets, and wherein each intervening elapsed time interval is multiple times greater in duration than the associated pulse set time intervals of the first pulse set and the additional pulse sets.

11. The method of claim 1, wherein the range information comprises an overall distance between the detector and the target.

12. The method of claim 1, further comprising using the range information to adjust a position of a moveable object.

13. An apparatus comprising:
an emitter configured to emit a set of pulses of electromagnetic radiation to illuminate a target downrange from the emitter, the set of pulses comprising a first emitted pulse with a first waveform characteristic and a second emitted pulse with a second waveform characteristic different from and in non-quadrature relation with the first waveform characteristic;
a detector configured to receive a reflected set of pulses from the target, the reflected set of pulses comprising a first received pulse corresponding to the first emitted pulse and a second received pulse corresponding to the second emitted pulse, the detector further configured to combine the first and second received pulses to determine range information associated with the target.

14. The apparatus of claim 13, wherein the range information comprises an overall distance between the detector and the target.

15. The apparatus of claim 13, further comprising a controller circuit which controllably positions a moveable element responsive to the range information.

16. The apparatus of claim 13, wherein the first and second waveform characteristics are each a phase of the respective first and second emitted pulses, wherein the second emitted pulse is out of phase with the first emitted pulse by a non-zero phase differential that is not a multiple of 90 degrees, and the range information is determined responsive to a detected phase differential between the first and second received pulses.

17. The apparatus of claim 16, wherein the non-zero phase differential between the first and second emitted pulses has a magnitude of nominally between 5 degrees and 30 degrees.

18. The apparatus of claim 13, wherein the detector is configured to determine the range information by calculating an average of respective phases of the first and second received pulses.

19. The apparatus of claim 13, wherein the pulse set is a first pulse set, wherein the emitter is further configured to emit a pulse set cycle comprising the first pulse set as well as a succession of additional, spaced apart pulse sets each having at least a first pulse and a second pulse, and wherein each pulse set in the pulse set cycle has a different phase differential between the associated first and second pulses in the corresponding pulse set.

20. The apparatus of claim 13, further comprising a programmable processor with associated program instructions stored in a memory configured to carry out at least selected operations of the emitter and the detector.
